(19) United States
(12) Patent Application Publication
(10) Pub. No.: US 2018/0362559 A1
(43) Pub. Date: Dec. 20, 2018

(54) BONE ACTIVE NITROGEN-CONTAINING BISPHOSPHONATES WITH A NEAR INFRARED FLUORESCENT LABEL

(71) Applicant: University of Southern California, Los Angeles, CA (US)

(72) Inventors: Charles E. MCKENNA, Los Angeles, CA (US); Boris A. KASHEMIROV, Los Angeles, CA (US); Kim NGUYEN, Los Angeles, CA (US)

(73) Assignee: University of Southern California, Los Angeles, CA (US)

(21) Appl. No.: 15/761,711

(22) PCT Filed: Sep. 21, 2016
(86) PCT No.: PCT/US16/52966

§ 371 Date: Mar. 20, 2018

Related U.S. Application Data
(60) Provisional application No. 62/221,601, filed on Sep. 21, 2015.

(51) Int. Cl. C07F 9/58; G01N 33/533; G01N 33/68; A61K 49/00

(52) U.S. Cl. CPC C07F 9/58; G01N 33/533; G01N 33/6893; G01N 2800/108; A61K 49/0043; A61K 49/0052; A61K 49/0032

(57) ABSTRACT

A compound of Formula I has the structure:

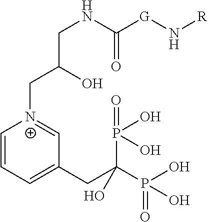

wherein G is a straight, branched or cyclic alkyl group having 2 to 50 carbon atoms or an ether group, and R is a fluorescent dye. In some embodiments, G is selected from the group consisting of —(CH₂)ₙ— and —(CH₂OCH₂)ₘ—, wherein n is an integer from 2 to 18, m is an integer from 2 to 18.

### RELATED APPLICATIONS

This application claims the benefit of U.S. Provisional Application No. 62/221,601, filed Sep. 21, 2015, the entire contents of which are incorporated herein by reference in its entirety.

### FIELD OF THE INVENTION

The present invention relates to bisphosphonate compounds and their use in imaging.

### BACKGROUND OF THE INVENTION

Imaging agents incorporating a targeting drug and visualizing moiety are indispensable in medical diagnostics and are invaluable aids in pharmacological drug development.¹⁻³ Fluorophores absorbing in the visible region and emitting in the visible-near IR have found increasing application in this area owing to their scanning accessibility, convenience of use and sensitivity to detection.⁴ 5- or 6-carboxyfluorescein (5-FAM, 6-FAM) and other fluorescent dyes are typically conjugated to a drug or protein by coupling of a substituent-CO₂H with a primary amino function promoted by a reagent such as DCC⁵ or by direct reaction of a primary amino group with the label's activated substituent-CO₂X (X═, e.g. succinimidyl, SE), thus forming an amide bond between the fluorescent label and the drug.⁶ However, in cases when the parent drug structure lacks a primary amine group, often a linker⁷ between the drug and label or structural modification to the drug⁸ is necessary for labeling. In general, amido links are preferable to esters which may be labile to hydrolysis in vitro or in vivo.

Bone-targeting methylenebisphosphonate drugs such as Risedronate (1-hydroxy-2-pyridin-3-ylethane-1,1-diyl)bis(phosphonic acid), Zoledronate [hydroxy(1H-imidazol-1-yl)methylene]bis(phosphonic acid), Ibandronate {-hydroxy-3-[methyl(pentyl)amino]propane-1,1-diyl}bis(phosphonic acid), Palmidronate (3-amino-1-hydroxypropane-1,1-diyl)bis(phosphonic acid), and Alendronate (4-amino-1-hydroxybutane-1,1-diyl)bis(phosphonic acid) are extensively used in the clinic to treat osteoporosis and other disorders of bone metabolism.^{9, 10} Some bisphosphonate drugs have been shown to inhibit metastasis in bone cancer, and also to exhibit an anti-neoplastic effect on bone tumors.^{11, 12} Methylenebisphosphonate drugs α-substituted with an aminoalkyl or N-containing heterocyclic group have been suggested to inhibit specifically one or more enzymes of the mevalonic pathway; the positive charge on nitrogen at physiological pH contribute to inhibitory potency and thus to the efficacy of this class of anti-osteoporotic drugs.¹³⁻¹⁸ In contrast, the bone affinity is almost solely determined by the bisphosphonate moiety itself.^{10, 18, 19} Bisphosphonates have the general structure:

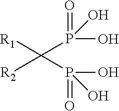

Fluorescently labeled bisphosphonate drugs are needed to improve understanding of bone distribution, cellular distribution, and cell absorption selectivity. The clinically significant but thus far poorly understood anti-metastatic and anti-tumor cell effects of some bisphosphonates offers a rationale for developing such imaging probes. Recent reports of a small number of previously unidentified osteonecrotic onsets that may be linked to prolonged therapy with at least one bisphosphonate also suggest an urgent requirement for improved understanding of bisphosphonate drug distribution in bone tissues.^{20, 21}

An Alexa Fluor 488 carboxamide conjugate of (4-amino-1-hydroxybutane-1,1-diyl)bis(phosphonic acid) (AF-ALN), linked the ε-amino group, was used to demonstrate imaging of this drug derivative, but the purity was apparently low.²² Amidization of aminoalkylidenebisphosphonates such as (4-amino-1-hydroxybutane-1,1-diyl)bis(phosphonic acid) abolishes the N atom positive charge which may contribute to drug inhibitory potency, as cited above. In addition, a comparable acylation approach for conjugating bisphosphonate drugs in which the N atom is contained in an aromatic ring such as the pyridyl group in risedronate is not advantageous, and no labeled version of risedronate has been available to date to the inventors' knowledge. Additional structural modifications to the parent drug are therefore required to introduce the appropriate chemical functionality and may also provide a “linker” region between the drug and label. The structure of risedronate, shown in its tetracid form, (1-hydroxy-2-pyridin-3-ylethane-1,1-diyl)bis(phosphonic acid), is:

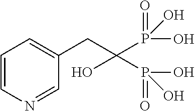 [SMILES: O=P(O)(O)C(O)(Cc1cccnc1)P(=O)(O)O]

### SUMMARY OF THE INVENTION

One aspect of the present invention is directed to compounds of Formula I.

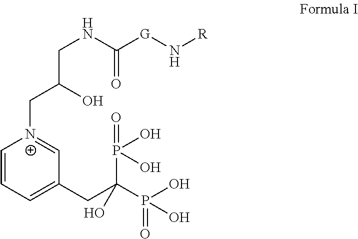

In Formula I, G is a straight, branched or cyclic alkyl group having 1 to 50 carbon atoms or an ether group, and R is a fluorescent dye. In some embodiments, G is selected from the group consisting of —(CH₂)ₙ— and —(CH₂OCH₂)ₘ—, wherein n is an integer from 2 to 18, m is an integer from 2 to 18. In some embodiments, n is an integer from 2 to 8, and m is an integer from 2 to 8. In some preferred embodiments, G may be —(CH₂)₂—, —(CH₂)₆— or —(CH₂OCH₂)₄—.

In one embodiment, the fluorescent dye may be 5-carboxyfluorescein (5-FAM), 6-FAM, Alexa Flu.

In one embodiment, the fluorescent dye may be 5-FAM, 6-FAM, Alexa Fluor 647 (AF647), or Sulfo-Cyanine5 (Sulfo-Cy5).

In another aspect of the present invention, the compounds of Formula I are used in a method to identify a bone disease or a bone condition in a subject. The method includes administering a pharmaceutical composition that includes Formula I to the subject, measuring the fluorescence in bone tissue or bone cells, and comparing the fluorescence to a control value of a subject without a bone disease and bone condition.

In one embodiment, the bone disease or bone condition includes osteoporosis, Paget's disease, metastatic bone cancers, hyperparathyroidism, rheumatoid arthritis, algodystrophy, stemo-costoclavicular hyperostosis, Gaucher's disease and Engleman's disease.

In another embodiment, the administering of the pharmaceutical composition includes the following routes of administration: intravenous, intradermal, subcutaneous, oral, transdermal, transmucosal, and rectal.

In another embodiment, the pharmaceutical composition further includes at least one excipient.

In another aspect of the present invention, the compounds of Formula I are used in a method of delivering imaging probes to sites of bone erosion. The method includes administering a pharmaceutical composition that includes Formula I to the subject, imaging the fluorescence, and comparing the fluorescence to a control value of a subject without bone erosion.

In one embodiment, imaging is visualized by confocal microscopy or near-infrared (NIR) fluorescence imaging systems.

Another aspect of the present invention is directed to compounds of Formula II and Formula III.

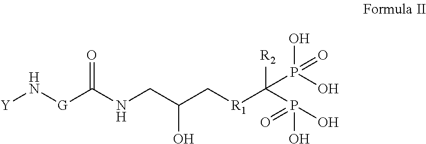

In Formula II, Y is a fluorescent dye, and G includes linear, branched, and cyclic alkyl chains, in which the number of carbon atoms is 1 to 8, and polyethyleneoxy units of formula (OCH₂CH₂)ₘ, wherein m is an integer from 1 to 3000. R₁ is a 3-ethyl-pyridine substituent or a 1-ethyl-1H-imidazole substituent. R₂ includes H, OH, F and Cl.

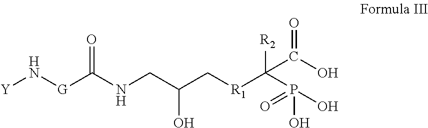

In Formula III, Y is a fluorescent dye, and G includes linear, branched, and cyclic alkyl chains, in which the number of carbon atoms is 1 to 8, and polyethyleneoxy units of formula (OCH₂CH₂)ₘ, wherein m is an integer from 1 to 3000. R₁ is a 3-ethyl-pyridine substituent or a 1-ethyl-1H-imidazole substituent. R₂ includes H, OH, F and Cl.

In one embodiment, the fluorescent dye may be 5-carboxyfluorescein (FAM), 6-FAM, Alexa Fluor 647 (AF647), or Sulfo-Cyanine5 (Sulfo-Cy5).

In another aspect of the present invention, the compounds of Formula II or III are used in a method to identify a bone disease or a bone condition in a subject. The method includes administering a pharmaceutical composition that includes Formula II or III to the subject, measuring the fluorescence in bone tissue or bone cells, and comparing the fluorescence to a control value of a subject without a bone disease and bone condition.

In one embodiment, the bone disease or bone condition includes osteoporosis, Paget's disease, metastatic bone cancers, multiple myeloma, hyperparathyroidism, rheumatoid arthritis, algodystrophy, stemo-costoclavicular hyperostosis, Gaucher's disease and Engleman's disease.

In another embodiment, the administering of the pharmaceutical composition includes the following routes of administration: intravenous, intradermal, subcutaneous, oral, transdermal, transmucosal, and rectal.

In another embodiment, the pharmaceutical composition further includes at least one excipient.

In another aspect of the present invention, the compounds of Formula II or III are used in a method of delivering imaging probes to sites of bone erosion. The method includes administering a pharmaceutical composition that includes Formula II or III to the subject, imaging the fluorescence, and comparing the fluorescence to a control value of a subject without bone erosion.

In one embodiment, imaging is visualized by confocal microscopy or near-infrared (NIR) fluorescence imaging systems.

Other aspects and advantages of the invention will be apparent from the following description and the appended claims.

### DETAILED DESCRIPTION OF THE INVENTION

As used herein the term “bone disease” or “bone condition” refers to or describes any affliction that involves the skeletal system and encompasses any condition that is associated with an impairment of the normal state of the skeletal system including congenital defects, pathological conditions such as cancer, and responses to environmental factors and infectious agents (bacterial, viral, etc.). Examples of bone diseases include but are not limited to osteoporosis, Paget's disease, metastatic bone cancers, multiple myeloma, hyperparathyroidism, rheumatoid arthritis, algodystrophy, stemo-costoclavicular hyperostosis, Gaucher's disease, Engleman's disease, disorders of bone metabolism, and the like.

As used herein, the term “subject” refers to any animal (e.g., a mammal), including, but not limited to, humans, non-human primates, rodents, and the like, which is to be the recipient of a particular treatment. Preferably the subject is a human.

The term “phosphonate” describes organic compounds containing one or more C—PO(OH)₂ or C—PO(OR)₂ (with R=alkyl, aryl) groups. The “phosphonate” as used herein preferably refers to analogs of phosphonate. Examples of phosphonates include but are not limited to bisphosphonates, phosphonoacetates, methylenebisphosphonates, phosphonocarboxylates, nitrogen-containing bisphosphonates, and the like.

Phosponates are preferably fluorescently labeled or conjugated with fluorophores or near-infrared agents. Examples of fluorophores or near-infrared agents include but are not limited to Alexa Fluor Dyes®, Cye dyes, IRDyes®, other fluorophores, near-infrared agents, and the like. More specifically, fluorophores refer to 5-carboxyfluorescein (5-FAM), 6-carboxyfluorescein (6-FAM), AMCA-X, Rhodamine Red-X, Alexa Fluor 647, and the like.

Fluorescently labeled phosphonates may be used to improve the understanding of drug bone distribution, cellular distribution, and cell absorption selectivity.

Furthermore, phosphonate conjugates or compounds may be used in a variety of ways. For example, conjugates or compounds may be used as a drug for the treatment of bone diseases or as a diagnostic for the detection of bone disease. Conjugates or compounds can also be used to study bone disease and the distribution of phosphonates in bone tissues, and bone cells.

To practice methods of treatment, fluorescently labeled phosphonate compounds are administered to a human or other mammal in need thereof a therapeutically effective amount of the compound. Indications appropriate to such treatment include bone diseases or bone conditions that include but are not limited to osteoporosis, Paget's disease, metastatic bone cancers, hyperparathyroidism, rheumatoid arthritis, algodystrophy, stemo-costoclavicular hyperostosis, Gaucher's disease, Engleman's disease, disorders of bone metabolism, and the like.

To practice methods relating to the study of bone disease or distribution of phosphonates in bone tissue or cells, fluorescently labeled compounds can be used in various model systems, including enzymatic and cellular assays as well as in vivo. For enzymatic studies, fluorescent compounds can be pre-incubated with enzyme, and the reaction products can be detected according to standard procedures. For imaging of compounds of distribution in cells, fluorescent compounds can be added to cell culture medium using standard methods known to those skilled in the art, and then visualized by methods such as confocal microscopy. For in vivo studies, compounds incorporating a near-IR imaging agent may be administered intravenously or by other appropriate means to the animal and subsequently visualized with MR fluorescence imaging systems. Alternatively, compounds containing fluorescent labels may be administered and the distribution of the compound in bone tissues or organs determined postmortem.

A noninvasive tool for diagnosis of erosions and evaluation of the response to therapies is therefore an unmet need. Bisphosphonates (BPs) including risedronate (RIS) and zoledronate (ZOL) have high affinity for bone, particularly at active turnover sites. Thus, BPs could be ideal carriers to deliver imaging probes to sites of bone erosion in cancer or RA patients, for example. Near infrared (NIR)-BP conjugates may be used to noninvasively identify bone erosions because of higher penetration depth and attenuation of light scattering. Interestingly, the 5,6-carboxyfluorescein (FAM)-conjugated BPs (FAM-RIS and FAM-ZOL) retain anti-prenylation/antiresorptive activity in J774 macrophages and in osteoclasts, but, no significant anti-prenylation activity is observed with the NIR-BP conjugates based on Alexa Fluor 647 (AF647), Cy5 and IRDye 800CW. To expand the utility of these NIR agents, a series of AF647-RIS conjugates were prepared in order to reduce fluorophore interference on cellular activity. Linker lengths were extended between the AF647 fluorescent label and the pyridyl moiety to increase the distance of the fluorescent component from the BP binding site.

To practice methods of treatment, fluorescently labeled phosphonate compounds are administered to a human or other mammal in need thereof a therapeutically effective amount of the compound. Indications appropriate to such treatment include bone diseases that include but are not limited to osteoporosis, Paget's disease, metastatic bone cancers, multiple myeloma, hyperparathyroidism, rheumatoid arthritis, algodystrophy, stemo-costoclavicular hyperostosis, Gaucher's disease, Engleman's disease, disorders of bone metabolism, and the like.

To practice methods relating to imaging and the study of bone disease or bone condition or distribution of phosphonates in bone tissue or cells, fluorescently labeled compounds can be used in various model systems, including enzymatic and cellular assays as well as in vivo. For enzymatic studies, fluorescent compounds can be pre-incubated with enzyme, and the reaction products can be detected according to standard procedures. For imaging of compounds of distribution in cells, fluorescent compounds can be added to cell culture medium using standard methods known to those skilled in the art, and then visualized by methods such as confocal microscopy. For in vivo studies, compounds incorporating a near-IR imaging agent may be administered intravenously or by other appropriate means to the animal and subsequently visualized with NIR fluorescence imaging systems. Alternatively, compounds containing fluorescent labels may be administered and the distribution of the compound in bone tissues or organs determined postmortem.

The following experimental details are provided in order to demonstrate and further illustrate certain embodiments and aspects of the present invention and are not to be construed as limiting the scope thereof.

In a first embodiment, a compound of the following general Formula I is described.

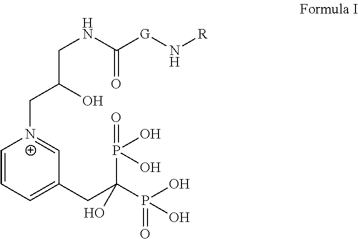

In Formula I, G is a straight, branched or cyclic alkyl group having 2 to 50 carbon atoms or an ether group, and R is a fluorescent dye. In some embodiments, G is selected from the group consisting of —(CH₂)ₙ— and —(CH₂OCH₂)ₘ—, wherein n is an integer from 2 to 18, m is an integer from 2 to 18. In some embodiments, n is an integer from 2 to 8, and m is an integer from 2 to 8. In some preferred embodiments, G may be —(CH₂)₂—, —(CH₂)₆— or —(CH₂OCH₂)₄—.

Compounds according to Formula I may be synthesized according to methods exemplified in Scheme I.

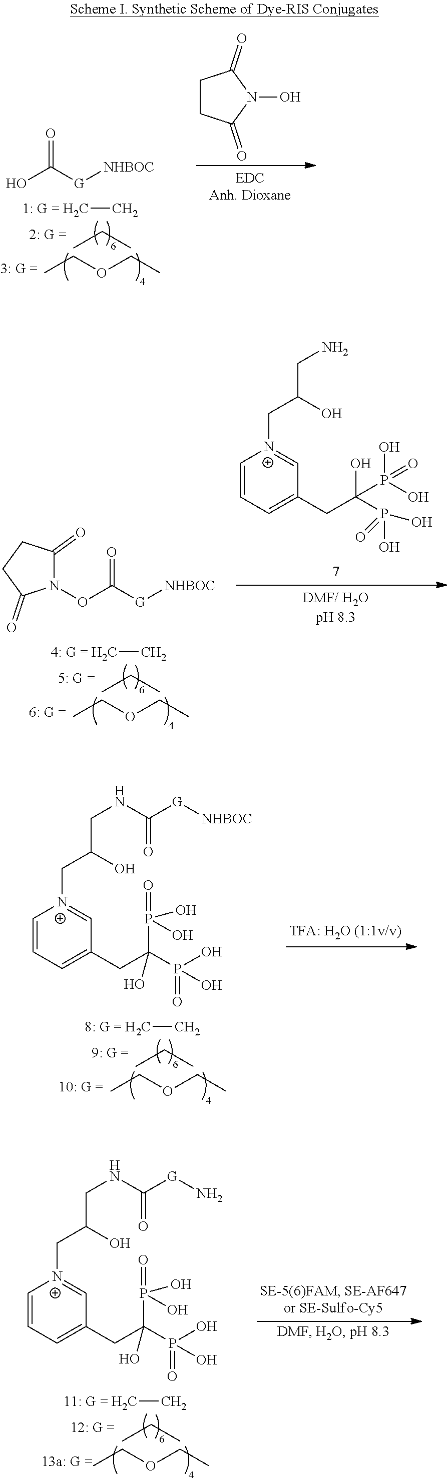

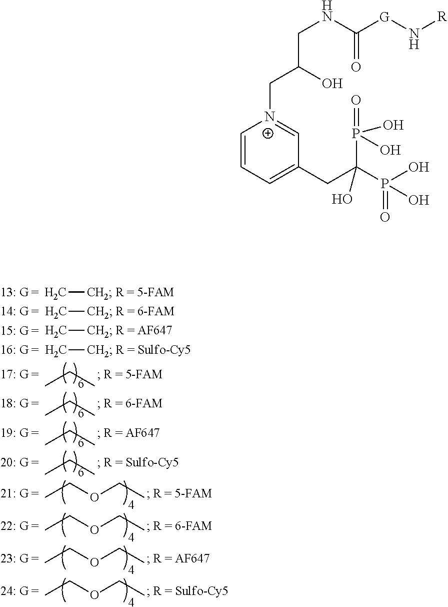

General Synthesis:

Succinimidyl ester activation BOC-protected acids were synthesized via reacting with N-hydroxysuccinimide (SuOH) and coupling reagents (e.g., EDC) in anhydrous solvents (e.g., DMF, or dioxane). The NHS activated acid was then reacted with Ris linker 7, which synthesis has been previously reported.²³ Deprotection of the newly synthesized extended rislinker (8, 9, 10) was performed using trifluoroacetic acid (TFA:water) (1:1 v/v) after SAX HPLC purification. The newly synthesized ExtRis linker (11, 12, 13a) was dissolved in HPLC water and pH was adjusted to 8.0-8.5. Dissolved in anhydrous DMF, SE-5(6)-carboxy fluorescein, SE-Sulfo-Cy5, or SE-AF647 was added dropwise into the ExtRis linker solution in order to obtain fluorescently labeled of ExtRis linker. The compounds labeled with 5(6)-carboxyfluorescein were purified using TLC and the two isomers were subsequently isolated via C18 HPLC while Alexa Fluor 647 and Sulfo-Cy5 labeled compounds were purified using only C18 HPLC.

5-carboxyfluorescein (FAM) has the following structure:

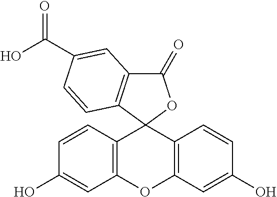 [SMILES: O=C(O)c1ccc2c(c1)C(=O)OC21c2ccc(O)cc2Oc2cc(O)ccc21]

6-carboxyfluorescein (FAM) has the following structure:

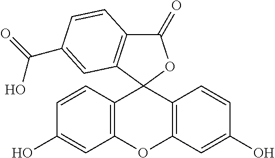 [SMILES: O=C(O)c1ccc2c(c1)C1(OC2=O)c2ccc(O)cc2Oc2cc(O)ccc21]

Alexa Fluor 647 has the following structure:

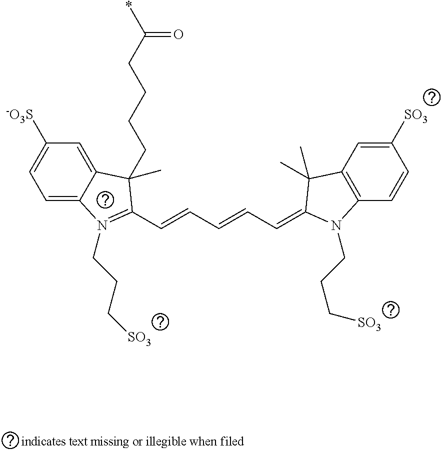

Sulfo-Cyanine5 carboxylic acid has the following structure:

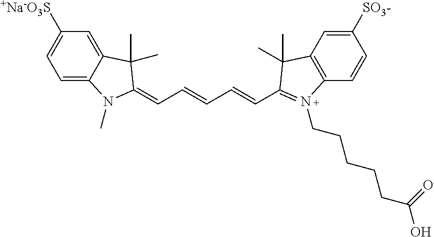

### 2.5-dioxopyrrolidin-1-yl 3-((tert-butoxycarbonyl)amino)propanoate (4)

In a dry flask containing a magnetic stir bar, 0.50 g of 1 (2.64 mmol) and 0.31 g of N-hydroxysuccinimide (SuOH) (2.72 mmol, 1.03 equivalent) were dissolved in 3 mL of distilled dioxane. Subsequently, 1.25 equivalent of 3-(ethyliminomethyleneamino)-N,N-dimethylpropan-1-amine in hydrogen chloride form (EDC) (0.63 g, 3.3 mmol) was added into the flask. The solution was cloudy, and 2 mL of freshly distilled dioxane was added into the reaction mixture. The reaction mixture was stirred under nitrogen for 30 minutes and then left for stirring overnight at room temperature. Dioxane was removed under vacuum. The residue was dissolved in chloroform and washed with water (3×). The organic layer was dried of over Na₂SO₄ and concentrated under vacuum. The desire product was precipitated in ethanol and dried in a desiccator, yielding 0.47 g of product (62%). The ¹H NMR spectral data matched the previously reported values for the compound.

### 2,5-dioxopyrrolidin-1-yl 7-((tert-butoxycarbonyl)amino)heptanoate (5)

In a dry flask containing a magnetic stir bar, 100.7 g of 2 (0.41 mmol) and 63.6 mg of N-hydroxysuccinimide (SuOH) (0.55 mmol, 1.25 equivalent) were dissolved in 3 mL of freshly distilled dioxane. Subsequently, 1.25 equivalent of 3-(ethyliminomethyleneamino)-N,N-dimethylpropan-1-amine in hydrogen chloride form (EDC) (106.5 mg, 0.55 mmol) was added into the flask. The solution was cloudy, and 2 mL of distilled dioxane was added into the reaction mixture. The reaction was stirred under nitrogen for 30 minutes and then left for stirring overnight at room temperature. Dioxane was removed under vacuum. The reaction mixture was dissolved in chloroform and washed with HPLC water (1×), followed by a wash with brine (1×). The organic layer was dried of over Na₂SO₄ and concentrated under vacuo, yielding 132 mg of crude product (62% yield based on ¹H NMR spectrum). The crude product was used for the subsequent reaction without purification.

### 2,5-dioxopyrrolidin-1-yl 2,2-dimethyl-4-oxo-3,8,11,14,17-pentaoxa-5-azaicosan-2-oate (6)

In a dry flask containing a magnetic stir bar, 41.6 mg of 3 (0.11 mmol) and 16.4 mg of N-hydroxysuccinimide (SuOH) (0.14 mmol, 1.25 equivalent) were dissolved in 1.5 mL of freshly distilled dioxane. Subsequently, 1.25 equivalent of 3-(ethyliminomethyleneamino)-N,N-dimethylpropan-1-amine (EDC) (27.35 mg, 0.14 mmol) was added into the flask. The reaction was stirred under nitrogen for an hour and then left for stirring overnight at room temperature. Solvent was removed and the reaction mixture was dissolved in chloroform, followed by washes with water (2×). The organic layer was dried over Na₂SO₄. Solvent was removed and the final product, as a colorless oil, was used without any additional purification for the next step.

### 1 (3-(3-((tert-butoxycarbonyl)amino)propanamido)-2-hydroxypropyl)-3-(2-hydroxy-2,2-diphosphonoethyl)pyridin-1-ium (8)

In a flask, 97.1 mg of 7 (0.197 mmol) was dissolved in 1.0 mL of HPLC water. The solution was adjusted to pH 8.3 using solid Na₂CO₃. In 0.5 mL of anhydrous DMF, 56.0 mg of 4 (0.195 mmol, 1.0 equivalent) was dissolved, which was then added dropwise into the solution of 7. The reaction was stirred overnight. Product 8 was obtained after SAX HPLC purification, yielding 66% (75.93 mg).

### 1-(3-(7-((tert-butoxycarbonyl)amino)heptanamido)-2-hydroxypropyl)-3-(2-hydroxy-2,2-diphosphonoethyl)pyridin-1-ium (9)

124.5 mg (0.22 mmol, 0.9 equivalent) of Compound 7 in the form of TEA/TFA salt, was dissolved in 1.0 mL of HPLC water, and pH was adjusted to 8.3 using solid Na₂CO₃. Compound S was dissolved in 300 μL anhydrous DMF and added dropwise into solution of 7. The reaction was left stirred overnight at room temperature. Product 9 was purified using SAX HPLC and obtained as TEA salt, yielding 110 mg (73%).

### 1-(23-hydroxy-2,2-dimethyl-4,20-dioxo-3,8,11,14,17-pentaoxa-5,21-diazatetracosan-24-yl)-3-(2-hydroxy-2,2-diphosphonoethyl)pyridin-1-ium (10)

In a flask, 69.16 mg of 6 (0.14 mmol), as trifluoroacetate sodium salts, was dissolved in 1.0 mL of HPLC water. The solution was adjusted to pH 8.3 using solid Na₂CO₃. Dissolved in 0.5 mL of anhydrous DMF, 6 was added dropwise into solution of 7 and stirred overnight. Purification of ExtRis-V2 was performed using preparative reverse-phase HPLC: Phenomenex Luna C18 (21.2 mm×250 mm, 5μ, 100 A pore size) column, flow rate 8.0 mL/min, gradient as follows: 0.1 N TEAB (pH 7.5) increasing to 8% of buffer B, 100% MeCN in 5 second, then staying at 8% of buffer B for 10 min, increasing to 25% of Buffer B for 5 min, followed by staying at 25% of buffer B for 25 min, and then staying at 50% of buffer B for 10 min. UV detection was set at 260 nm. Product 10 was obtained as triethylammonium salts, yielding 76% (69.6 mg).

### 1-(3-(3-aminopropanamido)-2-hydroxypropyl)-3-(2-hydroxy-2,2-diphosphonoethyl)pyridin-1-ium (11)

Product 8 was dissolved in HPLC water, and an equal volume of TFA was slowly added. The solution was stirred overnight at rt, giving quatitative yield of 11. The solvent was removed in vacuo, and the collected product was then used without further purification.

### 1-(3-(7-aminoheptanamido)-2-hydroxypropyl)-3-(2-hydroxy-2,2-diphosphonoethyl)pyridin-1-ium (12)

Product 9 was dissolved in HPLC water, and an equal volume of TFA was slowly added. The solution was stirred overnight at room temperature, giving quantitative yield of 12. The solvent was removed in vacuo, and the collected product was then used without further purification.

### 1-(1-amino-18-hydroxy-15-oxo-3,6,9,12-tetraoxa-16-azanonadecan-19-yl)-3-(2-hydroxy-2,2-diphosphonoethyl)pyridin-1-ium (13a)

Product 10 was dissolved in HPLC water, and an equal volume of TFA was slowly added. The solution was stirred overnight at rt, giving quantitative yield of 13a (79.4 mg, as triethylammonium trifluoroacetate salts). The solvent was removed in vacuo, and the collected product was then used without further purification.

### 5-FAM-ExtRis-V1 (13) (1-(3-(3-(3-carboxy-4-(6-hydroxy-1-oxo-3H-xanthen-9-yl)benzamido)propanamido)-2-hydroxypropyl)-3-(2-hydroxy-2,2-diphosphonoethyl)pyridin-1-ium) and 6-FAm-ExtRis-V1 (14) (1-(3-(3-(4-carboxy-3-(6-hydroxy-3-oxo-3H-xanthen-9-yl)benzamido)propanamido)-2-hydroxypropyl)-3-(2-hydroxy-2,2-diphosphonoethyl)pyridin-1-ium)

In 0.5 ml HPLC water, 75.9 mg of 11 (0.11 mmol) was dissolved and pH was adjusted to 8.30 using solid Na₂CO₃. 5(6)-Carboxyfluorescein, N-hydroxysuccinimide ester (a mixture of 5-{[(2,5-dioxopyrrolidin-1-yl)oxy]carbonyl}-2-(6-hydroxy-3-oxo-3H-xanthen-9-yl)benzoic acid and 4-{[(2,5-dioxopyrrolidin-1-7yl)oxy]carbonyl}-2-(6-hydroxy-3-oxo-3H-xanthen-9-yl)benzoic acid, 11.63 mg (0.025 mmol, 4 equivalent) was dissolved in anhydrous DMF (˜50 mg per 200 μL) and added dropwise into solution of 5. The solution became dark orange and some solid precipitate appeared. The pH of the solution was adjusted again using solid Na₂CO₃, dissolving all the precipitate. The reaction was stirred overnight. The unreacted dye was removed via purification by TLC with 100% MeOH as the eluant. The FAM-labeled compound stayed at the origin while the dye moved upward in the TLC plates, giving a yellow upper band, while the phosphonate-containing compounds remain at the origin. The desire product were extracted with HPLC water from the silica, centrifuged, and concentrated in vacuo to yield a dark red-orange solid. The compound was then dissolved in water and filtered through Nanosep 30K Omega filter. Purification of 5,6-FAM-ExtRis-V1 was performed using semi-preparative reverse-phase HPLC: Beckman C18 (10 mm×250 mm, 5μ, 100 A pore size) column, flow rate 4.0 mL/min, gradient as follows: 10% MeOH 0.1 N TEAC (pH 7) increasing to 40% of buffer B, 75% MeOH 0.1 N TEAC (pH 7.8), in 25 min, then increasing to 100% of Buffer B in 100 min. UV detection was set at 260 nm for the first 10 min and at 493 nm for the rest of the run. The final amount of labeled product is calculated from the UV absorption spectrum taking ε=73000 M⁻¹ cm⁻¹ in 1×PBS buffer at pH 7.4 and the isolated 13 and 14 are lyophilized, yielding a red-orange solids.

### AF647-ExtRis-V1 (15)

In 0.5 ml HPLC water, 16.2 mg of 11 as triethylammonium salt (0.03 mmol) was dissolved and pH was adjusted to 8.30 using solid Na₂CO₃. Alexa Fluor 647, succinimidyl ester, 1.0 mg (1 μmol, 0.1 equivalent) was dissolved in 150 μL anhydrous DMF and added dropwise into solution of 11. The solution became dark blue. The reaction was stirred overnight. Purification AF647-labeled compound, AF647-ExtRis-V1, was performed using semi-preparative reverse-phase HPLC: Beckman C18 (10 mm×250 mm, 5μ, 100 A pore size) column, flow rate 4.0 mL/min, gradient as follows: 10% MeOH 0.1 N TEAC (pH 7) increasing to 50% of buffer B, 75% MeOH 0.1 N TEAC (pH 7.8), in 25 min, then remained at 50% of Buffer B for 60 min. UV detection was set at 260 nm for the first 10 min and at 598 nm for the rest of the run. The final amount of labeled product is calculated from the UV absorption spectrum taking ε=239000 M⁻¹ cm⁻¹ in 1×PBS buffer at pH 7.4 and the isolated 15 is lyophilized, yielding a dark blue solid (47.9%).

### Sulfo-Cy5-ExtRis-V1 (16)

In 0.6 ml HPLC water, 12.7 mg of 11 as TEA and TFA salt (0.022 mmol) was dissolved and pH was adjusted to 8.30 using solid Na₂CO₃. Sulfo-Cy5, succinimidyl ester, 5.0 mg (6.6 μmol, 0.3 equivalent) was dissolved in 150 μL anhydrous DMF and added dropwise into solution of 11. The solution became dark blue. The reaction was stirred overnight. Purification Sulfo-Cy5-labeled compound 16 was performed using semi-preparative reverse-phase HPLC with Phenomenex C18 (10 mm×250 mm, 5μ, 100 A pore size) column, flow rate 4.0 mL/min, with a system of buffers, including 10% MeCN 0.1 N TEAC (pH 7.0) as buffer A and 75% MeCN 0.1 N TEAC (pH 7.8) as buffer B. UV detection was set at 260 nm for the first 10 min and at 598 nm for the rest of the run. The final amount of labeled product is calculated from the UV absorption spectrum taking s=271000 M⁻¹ cm⁻¹ at maximum absorption in 1×PBS buffer at pH7.4 and the final product 16 is lyophilized, yielding a dark blue solid (40.8%).

### 5-FAM-ExtRis-M (17) and 6-FAM-ExtRis-M (18)

In 0.8 ml HPLC water, 20 mg of 12 (0.029 mmol) was dissolved and pH was adjusted to 8.30 using solid Na₂CO₃. 5(6)-Carboxyfluorescein, N-hydroxysuccinimide ester (a mixture of 5-{[(2,5-dioxopyrrolidin-1-yl)oxy]carbonyl}-2-(6-hydroxy-3-oxo-3H-xanthen-9-yl)benzoic acid and 4-{[(2,5-dioxopyrrolidin-1-7yl)oxy]carbonyl}-2-(6-hydroxy-3-oxo-3H-xanthen-9-yl)benzoic acid, 31.6 mg (0.067 mmol, 2 equivalent) was dissolved in 600 μL anhydrous DMF and added dropwise into solution of 12. The solution became dark orange and some solid precipitate appeared. The pH of the solution was adjusted again using solid Na₂CO₃, dissolving all the precipitate. The reaction was stirred overnight. The unreacted dye was removed via purification by TLC with 100% MeOH as the eluant. The FAM-labeled compound stayed at the origin while the dye moved upward in the TLC plates, giving a yellow upper band, while the phosphonate-containing compounds remain at the origin. The desire product were extracted with HPLC water from the silica, centrifuged, and concentrated in vacuo to yield a dark red-orange solid. The compound was then dissolved in water and filtered through Nanosep 30K Omega filter. Purification and isolation of compound 17 and 18 were performed using semi-preparative reverse-phase HPLC with Phenomenex C18 (10 mm×250 mm, 5μ, 100 A pore size) column, flow rate 4.0 mL/min, with a system of buffers, including 10% MeCN 0.1 N TEAC (pH 7.0) as buffer A and 75% MeCN 0.1 N TEAC (pH 7.8) as buffer B. UV detection was set at 260 nm for the first 10 min and at 493 nm for the rest of the run. The final amount of labeled product is calculated from the UV absorption spectrum taking ε=73000 M⁻¹ cm⁻¹ at maximum absorption in 1×PBS buffer at pH 7.4 and the isolated final product 17 and 18 is lyophilized, yielding an orange solid (yield is not yet available).

### AF647-ExtRis-M (19)

In 0.3 ml HPLC water, 3.0 mg of 12 as TEA and TFA salt (4.3 μmol) was dissolved and pH was adjusted to 8.30 using solid Na₂CO₃. Sulfo-Cy5, succinimidyl ester, 5.0 mg (1.0 μmol, 0.3 equivalent) was dissolved in 50 μL anhydrous DMF and added dropwise into solution of 12. The solution became dark blue. The reaction was stirred overnight. Purification of compound 19 was performed using semi-preparative reverse-phase HPLC with Phenomenex C18 (10 mm×250 mm, 5μ, 100 A pore size) column, flow rate 4.0 mL/min, with a system of buffers, including 10% MeCN 0.1 N TEAC (pH 7.0) as buffer A and 75% MeCN 0.1 N TEAC (pH 7.8) as buffer B. UV detection was set at 260 nm for the first 10 min and at 598 nm for the rest of the run. The final amount of labeled product is calculated from the UV absorption spectrum taking ε=239000 M⁻¹ cm⁻¹ at maximum absorption in 1×PBS buffer at pH 7.4 and the final product 19 is lyophilized, yielding a dark blue solid (48.1%).

### Sulfo-Cy5-ExtRis-M (20)

In 0.5 ml HPLC water, 14.0 mg of 12 as TEA and TFA salt (0.020 mmol) was dissolved and pH was adjusted to 8.30 using solid Na₂CO₃. Sulfo-Cy5, succinimidyl ester, 5.0 mg (6.6 μmol, 0.3 equivalent) was dissolved in 150 μL anhydrous DMF and added dropwise into solution of 12. The solution became dark blue. The reaction was stirred overnight. Purification Sulfo-Cy5-labeled compound 20 was performed using semi-preparative reverse-phase HPLC with Phenomenex C18 (10 mm×250 mm, 5μ, 100 A pore size) column, flow rate 4.0 mL/min, with a system of buffers, including 10% MeCN 0.1 N TEAC (pH 7.0) as buffer A and 75% MeCN 0.1 N TEAC (pH 7.8) as buffer B. UV detection was set at 260 nm for the first 10 min and at 598 nm for the rest of the run. The final amount of labeled product is calculated from the UV absorption spectrum taking ε=271000 M⁻¹ cm⁻¹ at maximum absorption in 1×PBS buffer at pH 7.4 and the final product 20 is lyophilized, yielding a dark blue solid (50.4%).

### 5-FAM-ExtRis-V2 (21) (1-(1-(3-carboxy-4-(6-hydroxy-3-oxo-3H-xanthen-9-yl)phenyl)-20-hydroxy-1,17-dioxo-5,8,11,14-tetraoxa-2,18-diazahenicosan-21-yl)-3-(2-hydroxy-2,2-diphosphonoethyl)pyridin-1-ium) and 6-FAm-ExtRis-V2 (22) 1-(1-4-carboxy-3-(6-hydroxy-3-oxo-3H-xanthen-9-yl)phenyl)-20-hydroxy-1,17-dioxo-5,8,11,14-tetraoxa-2,18-diazahenicosan-21-yl)-3-(2-hydroxy-2,2-diphosphonoethyl)pyridin-1-ium

In 0.5 ml HPLC water, 12.1 mg of 13a (0.02 mmol) was dissolved and pH was adjusted to 8.30 using solid Na₂CO₃. 5(6)-Carboxyfluorescein, N-hydroxysuccinimide ester (a mixture of 5-{[(2,5-dioxopyrrolidin-1-yl)oxy]carbonyl)}-2-(6-hydroxy-3-oxo-3H-xanthen-9-yl)benzoic acid and 4-{[(2,5-dioxopyrrolidin-1-7yl)oxy]carbonyl}-2-(6-hydroxy-3-oxo-3H-xanthen-9-yl)benzoic acid, 9.85 mg (0.02 mmol, 1 equivalent) was dissolved in anhydrous DMF (˜50 mg per 200 μL) and added dropwise into solution of 13a. The solution became dark orange and some solid precipitate appeared. The pH of the solution was adjusted again using solid Na₂CO₃, dissolving all the precipitate. The reaction was stirred overnight. The unreacted dye was removed via purification by TLC with 100% MeOH as the eluant. The FAM-labeled compound stayed at the origin while the dye moved upward in the TLC plates, giving a yellow upper band, while the phosphonate-containing compounds remain at the origin. The desire product were extracted with HPLC water from the silica, centrifuged, and concentrated in vacuo to yield a dark red-orange solid. The compound was then dissolved in water and filtered through Nanosep 30K Omega filter. Purification of 5,6FAM-ExtRis-V2 was performed using semi-preparative reverse-phase HPLC: Beckman C18 (10 mm×250 mm, 5μ, 100 A pore size) column, flow rate 4.0 mL/min, gradient as follows: 10% MeOH 0.1 N TEAC (pH 7) increasing to 40% of buffer B, 75% MeOH 0.1 N TEAC (pH 7.8), in 25 min, then increasing to 100% of Buffer B in 100 min. UV detection was set at 260 nm for the first 10 min and at 493 nm for the rest of the run. The final amount of labeled product is calculated from the UV absorption spectrum taking ε=73000 M⁻¹ cm⁻¹ in 1×PBS buffer at pH7.4 and the isolated 21 and 22 are lyophilized, yielding a red-orange solids (16.1% and 17.6 for 6 and 5 isomers, respectively).

### AF647-ExtRis-V2 (23)

In 0.5 ml HPLC water, 7.35 mg of 13a as triethylammonium salt (0.01 mmol) was dissolved and pH was adjusted to 8.30 using solid Na₂CO₃. Alexa Fluor 647, succinimidyl ester, 1.0 mg (1 μmol, 10 equivalent) was dissolved in 200 μL anhydrous DMF and added dropwise into solution of 13a. The solution became dark blue. The reaction was stirred overnight. Purification AF647-labeled compound, AF647-ExtRis-V2, was performed using semi-preparative reverse-phase HPLC: Beckman C18 (10 mm×250 mm, 5μ, 100 A pore size) column, flow rate 4.0 mL/min, gradient as follows: 10% MeOH 0.1 N TEAC (pH 7) increasing to 50% of buffer B, 75% MeOH 0.1 N TEAC (pH 7.8), in 25 min, then remained at 50% of Buffer B for 60 min. UV detection was set at 260 nm for the first 10 min and at 598 nm for the rest of the run. The final amount of labeled product is calculated from the UV absorption spectrum taking ε=239000 M⁻¹ cm⁻¹ in 1×PBS buffer at pH 7.4 and the isolated 23 is lyophilized, yielding a dark blue solid (20.3%).

### Sulfo-Cy5-ExtRis-V2 (24)

In 0.5 ml HPLC water, 14.0 mg of 13a as TEA and TFA salt (0.017 mmol) was dissolved and pH was adjusted to 8.30 using solid Na₂CO₃. Sulfo-Cy5, succinimidyl ester, 5.0 mg (6.6 μmol, 0.3 equivalent) was dissolved in 150 μL anhydrous DMF and added dropwise into solution of 13a. The solution became dark blue. The reaction was stirred overnight. Purification Sulfo-Cy5-labeled compound 24 was performed using semi-preparative reverse-phase HPLC with Phenomenex C18 (10 mm×250 mm, 5μ, 100 A pore size) column, flow rate 4.0 mL/min, with a system of buffers, including 10% MeCN 0.1 N TEAC (pH 7.0) as buffer A and 75% MeCN 0.1 N TEAC (pH 7.8) as buffer B. UV detection was set at 260 nm for the first 10 min and at 598 nm for the rest of the run. The final amount of labeled product is calculated from the UV absorption spectrum taking ε=271000 M⁻¹ cm⁻¹ at maximum absorption in 1×PBS buffer at pH 7.4 and the final product 24 is lyophilized, yielding a dark blue solid.

### Compounds and In Vitro Activities

To determine if these AF-BP structural modifications improved the osteoclast (OC) inhibitory activity, these modifications were tested in vitro using mouse bone marrow cells. It was found that while the control probe AF647-RIS had little effect on OC numbers (221+/−37, 241+/−21 and 230+/−36 for 0.1, 1 and 10 uM AF647-RIS), two linker extensions significantly decreased OC numbers: 218+/−24, 208+/−21 and 19+/−5 for 0.1, 1 and 10 uM AF647-RIS-v1 (a 3 carbon extension), and 205+/−22, 221+/−19 and 43+/−10 for 0.1, 1 and 10 uM AF647-RIS-v2 (a 15-atom poly(ethylene glycol) linker), compared to vehicle (239+/−17) and RIS (214+/−37, 220+/−28 and 143+/−21 for 0.1, 1 and 10 uM). These data suggest that increasing the linker chain length of an AF647-BP conjugate can dramatically increase its inhibitory effects. Thus, while the pharmacologically inactive AF647-RIS may be a useful tool to detect bone erosion in RA, longer linked antiresorptive conjugates (AF647-RIS-V1/V2) may be used to study the location and therapeutic effect of N-BPs.

In vitro studies indicated that the following compounds were the most active in osteoclast culture and bone slice resorption assays: 13, 14, 15, 21, 22, and 23.

In a second embodiment, a compound of the following general Formula II is described.

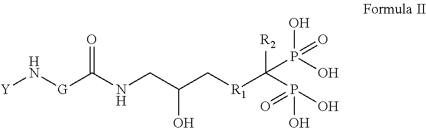

In Formula II, Y is a fluorescent dye, and G includes linear, branched, and cyclic alkyl chains, in which the number of carbon atoms is 1 to 8, and polyethyleneoxy units of formula (OCH₂CH₂)ₘ, wherein m is an integer from 1 to 3000. R₁ is a 3-ethyl-pyridine substituent or a 1-ethyl-1H-imidazole substituent. R₂ includes H, OH, F and Cl.

Compounds according to Formula II may be synthesized as exemplified in Scheme II.

### Scheme II. Synthetic Scheme of New Dye-BP Conjugates

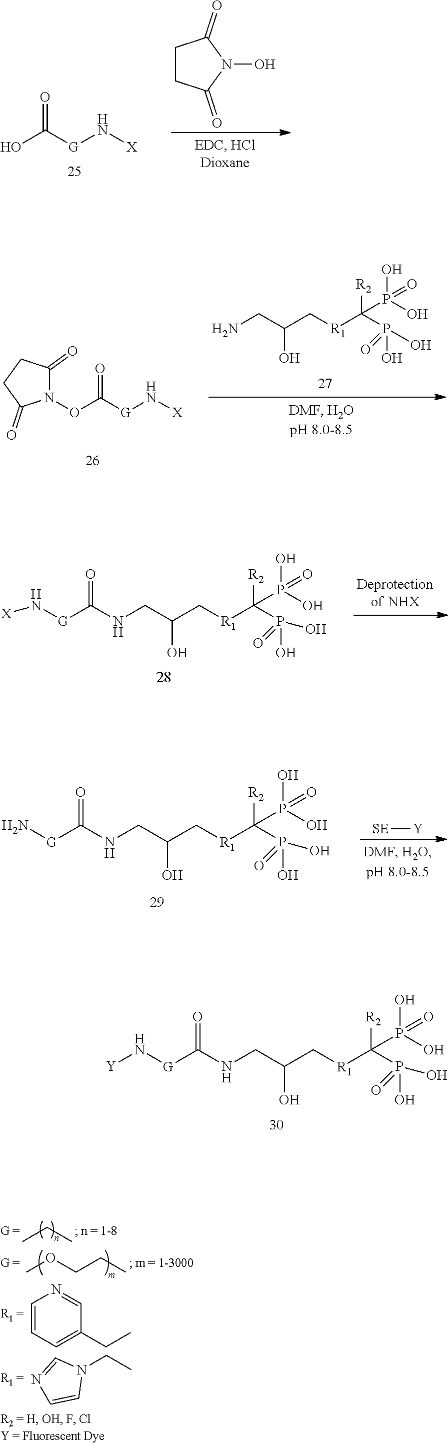

Scheme II describes the general route for the synthesis of the new dye-BP conjugates. The linker G used in this scheme includes both hydrophobic and hydrophilic linkers. The hydrophobic linker contains either linear, branched, or cyclic alkyl chains having from n=1-8 carbon atoms. The hydrophilic linker consists of a polyethyleneoxy unit of formula (OCH₂CH₂)ₘ, wherein m is an integer from 1 to about 3000, or combinations of polyethyleneoxy units of formula (OCH₂CH₂)ₘ with integrated amide or ester groups.

The protecting group X for the amino group may be tBOC but a so can be other groups, such as Fmoc, Cbz, Bn, Mtt, ortho-nitrobenzyl or other commonly used protecting groups for the amino group.

The bisphosphonate (BP) compounds used for this synthesis can have either 3-ethyl-pyridine or 1-ethyl-1H-imidazole substituents at the R₁ position, or with any other attaching positions on the heterocyclic rings, in combination with R₂═H.

This general synthesis is applicable to a wide range of succinimidyl ester form of fluorescent end near IR dyes, including 5,6-FAM, 5,6-ROX, RRX, BDP, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa, Fluor 610, Alexa Fluor 633, Alexa Fluor 635, Alexa Fluor 647, Alexa Fluor 660, Alexa Fluor 680, Alexa Fluor 700, Alexa Fluor 750, Alexa Fluor 790, IR 800CW, Cy3, Cy3,5, Sulfa-Cy3, Cy5, Cy5.5, Sulfo-Cy5, Cy7, Cy7.5, Sulfa-Cy7.

In a third embodiment, a compound of the following general Formula III is described.

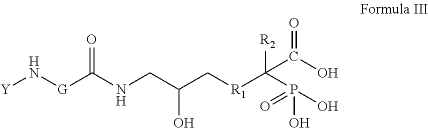

In Formula III, Y is a fluorescent dye, and G includes linear, branched, and cyclic alkyl chains, in which the number of carbon atoms is 1 to 8, and polyethyleneoxy units of formula (OCH₂CH₂)ₘ, wherein m is an integer from 1 to 3000. R₁ is a 3-ethyl-pyridine substituent or a 1-ethyl-1H-imidazole substituent. R₂ includes H, OH, F and Cl.

The methods described above can also be applied to phosphonocarboxylate (PC) compounds, illustrated in Scheme III (below).

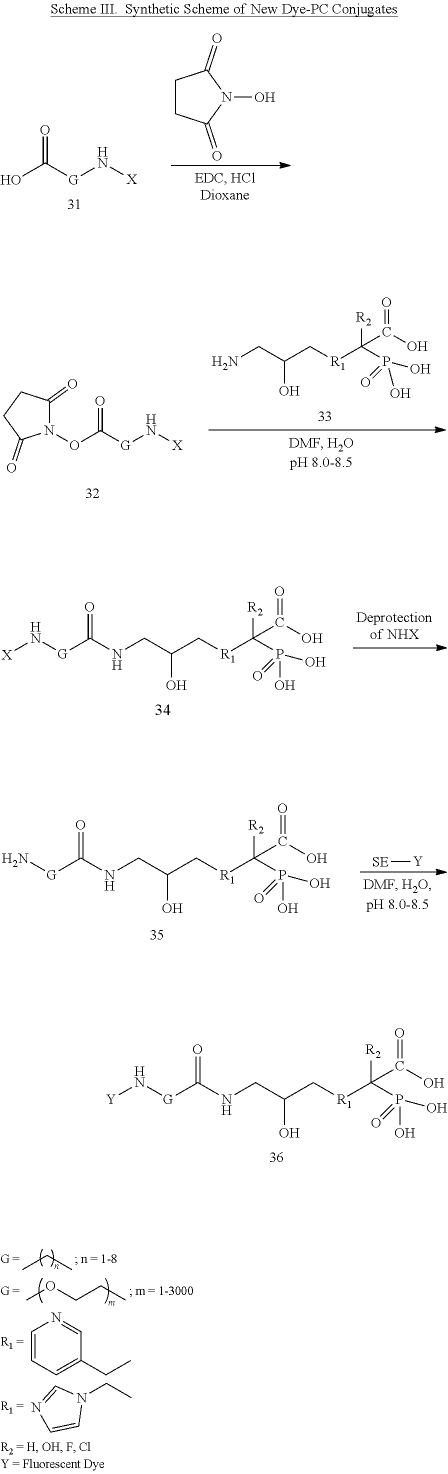

### General Synthesis

Succinimidyl ester activation of BOC-protected acids 31 were synthesized via reacting with N-hydroxysuccinimide (SuOH) and coupling reagents (e.g., EDC) in anhydrous solvents (e.g., DMF, or dioxane). In step 2, the NHS activated acid 32 was then reacted with PC-linker 33, which synthesis has been previously reported.²³ Deprotection of the newly synthesized extended BP-linker 34 (ExtPC-linker) was performed using appropriate deprotection method for different protecting groups after SAX HPLC purification. The newly synthesized ExtPC-linker 35 was dissolved in HPLC grade water and pH was adjusted to 8.0-8.5. Dissolved in anhydrous DMF, the NHS activated fluorescent dye was added dropwise into the ExtPC linker solution in order to obtain fluorescently labeled of ExtPC-linker 36. The compounds labeled with fluorescent dyes were purified using TLC and reverse phase C18 HPLC.

Fluorescently labeled or conjugated phosphonates or compounds of the invention are formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

The compounds can be contained in tablets, troches, pills, capsules, and the like, which may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

In one embodiment, the compounds are prepared with carriers that will protect the compounds against rapid elimination from the body, such as a controlled release formulation, including implants and microencapsulated delivery systems. Therapeutic agents, may also comprise siRNAs conjugated to cationic polypeptides, amphipathic compounds, polycations, liposomes or PEGlyated liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

### REFERENCES

All references cited herein, including those below and including but not limited to all patents, patent applications, and non-patent literature referenced below or in other portions of the specification, are hereby incorporated by reference herein in their entirety.
[1] Giepmans, B. N., Adams, S. R., Ellisman, M. H., and Tsien, R. Y. (2006) The fluorescent toolbox for assessing protein location and function, Science 312, 217-224.
[2] Gumbleton, M., and Stephens, D. J. (2005) Coming out of the dark: the evolving role of fluorescence imaging in drug delivery research, Adv Drug Deliv Rev 57, 5-15.
[3] Stephens, D. J., and Allan, V. J. (2003) Light microscopy techniques for live cell imaging, Science 300, 82-86.
[4] Waggoner, A. (2006) Fluorescent labels for proteomics and genomics, Curr. Opin. Chem. Biol. 10, 62-66.
[5] Adamczyk, M., Fishpaugh, J. R., and Heuser, K. J. (1997) Preparation of succinimidyl and pentafluorophenyl active esters of 5- and 6-carboxyfluorescein, Bioconjug Chem 8, 253-255.
[6] Li, L., Kracht, J., Peng, S., Bernhardt, G., Elz, S., and Buschauer, A. (2003) Synthesis and pharmacological activity of fluorescent histamine H2 receptor antagonists related to potentidine, Bioorg. Med Chem. Lett. 13, 1717-1720.
[7] Bertrand, R., Derancourt, J., and Kassab, R. (2000) Fluorescence characterization of structural transitions at the strong actin binding motif in skeletal myosin affinity labeled at cysteine 540 with novel spectroscopic cysteaminyl mixed disulfides, Biochemistry 39, 14626-14637.
[8] Ung, A. T., and Pyne, S. G. (1996) Synthesis of fluorescent and biotinylated analogues of (1R, 2S, 3R)-2-acetyl-4(5)-(1,2,3,4-tetrahydroxybutyl)imidazole, Tetrahedron Lett. 37, 6209-6212.
[9] Rodan, G. A., and Martin, T. J. (2000) Therapeutic approaches to bone diseases, Science 289, 1508-1514.
[10] Russell, R. G., and Rogers, M. J. (1999) Bisphosphonates: from the laboratory to the clinic and back again, Bone 25, 97-106.
[11] Bagi, C. M. (2005) Targeting of therapeutic agents to bone to treat metastatic cancer, Adv Drug Deliv Rev 57, 995-1010.
[12] Clezardin, P., Ebetino, F. H., and Fournier, P. G. (2005) Bisphosphonates and cancer-induced bone disease: beyond their antiresorptive activity, Cancer Res. 65, 4971-4974.
[13] Cheng, F., and Oldfield, E., (2004) Inhibition of isoprene biosynthesis pathway enzymes by phosphonates, bisphosphonates, and diphosphates, J. Med. Chem. 47, 5149-5158.
[14] Ebetino, F. H., Roze, C. N., McKenna, C. E., Barnett, B. L., Dunford, J. E., Russell, R. G. G., Mieling, G. E., and Rogers, M. J. (2005) Molecular interactions of nitrogen-containing bisphosphonates within farnesyl diphosphate synthase, J. Organomet. Chem. 690, 2679-2687.
[15] Kavanagh, K. L., Guo, K., Dunford, J. E., Wu, X., Knapp, S., Ebetino, F. H., Rogers, M. J., Russell, R. G., and Oppermann, U. (2006) The molecular mechanism of nitrogen-containing bisphosphonates as antiosteoporosis drugs, Proc Natl Acad Sci USA 103, 7829-7834.
[16] Kotsikorou, E., and Oldfield, E. (2003) A quantitative structure-activity relationship and pharmacophore modeling investigation of aryl-X and heterocyclic bisphosphonates as bone resorption agents, J. Med. Chem. 46, 2932-2944.
[17] Martin, M. B., Arnold, W., Heath, H. T., 3rd, Urbina, J. A., and Oldfield, E. (1999) Nitrogen-containing bisphosphonates as carbocation transition state analogs for isoprenoid biosynthesis, Biochem. Biophys. Res. Commun. 263, 754-758.
[18] Nancollas, G. H., Tang, R., Phipps, R. J., Henneman, Z., Guide, S., Wu, W., Mangood, A., Russell, R. G. G., and Ebetino, F. H. (2006) Novel insights into actions of bisphosphonates on bone: Differences in interactions with hydroxyapatite, Bone 38, 617-627.
[19] van Sonsbeek, S., Pullens, B., and van Benthem, P. P. (2015) Positive pressure therapy for Meniere's disease or syndrome, Cochrane Database Syst Rev 3, CD008419.
[20] Mignogna, M. D., Lo Russo, L., Fedele, S., Ciccarelli, R., and Lo Muzio, L. (2006) Case 2. Osteonecrosis of the jaws associated with bisphosphonate therapy, Section of Oral Medicine, Dept of Odontostomatological and Maxillofacial Sciences, University Federico II, Naples, Italy: United States, 1475-1477.
[21] Schirmer, I., Peters, H., Reichart, P. A., and Durkop, H. (2005) Bisphosphonates and osteonecrosis of the jaw., Mund-, Kiefer-und Gesichtschirurgie: MKG 9, 239-245.
[22] Thompson, K., Rogers, M. J., Coxon, F. P., and Crockett, J. C. (2006) Cytosolic entry of bisphosphonate drugs requires acidification of vesicles after fluid-phase endocytosis, Mol. Pharmacol. 69, 1624-1632.
[23] Kashemirov, B. A., Bala, J. L., Chen, X., Ebetino, F. H., Xia, Z., Russell, R. G., Coxon, F. P., Roelofs, A. J., Rogers, M. J., and McKenna, C. E. (2008) Fluorescently labeled risedronate and related analogues: “magic linker” synthesis, Bioconjug Chem 19, 2308-2310.

What is claimed is:

1. A compound selected from the group consisting of Formula I, 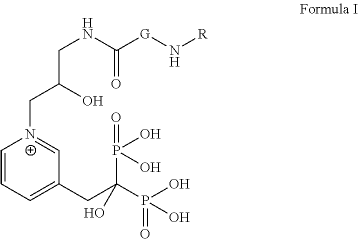
wherein G is selected from the group consisting of —(CH₂)ₙ— and —(CH₂OCH₂)ₘ—,
wherein n is an integer from 2 to 18, m is an integer from 2 to 18, and
R is a fluorescent dye.

2. The compound of claim 1, wherein G is selected from the group consisting of —(CH₂)₂—, —(CH₂)₆—, and —(CH₂OCH₂)₆—.

3. The compound of claim 1, wherein R is selected from the group consisting of 5-carboxyfluorescein (5-FAM), 6-FAM, Alexa Fluor 647 (AF647), and Sulfo-Cyanine5 (Sulfo-Cy5).

4. A method of noninvasively identifying a bone disease or a bone condition in a subject comprising:
administering a pharmaceutical composition comprising a compound of claim 1 to the subject;
measuring the fluorescence in bone tissue or bone cells; and
comparing the fluorescence to a control value of a subject without a bone disease and bone condition.

5. The method of claim 4, wherein the bone disease or bone condition is selected from the group consisting of osteoporosis, Paget's disease, metastatic bone cancers, multiple myeloma, hyperparathyroidism, rheumatoid arthritis, algodystrophy, stemo-costoclavicular hyperostosis, Gaucher's disease and Engleman's disease.

6. The method of claim 4, wherein the administering of the pharmaceutical composition is selected from the route of administration consisting of intravenous, intradermal, subcutaneous, oral, transdermal, transmucosal, and rectal.

7. The method of claim 4, wherein the pharmaceutical composition further comprises at least one excipient.

8. A method of delivering imaging probes to sites of bone erosion comprising:
administering a pharmaceutical composition comprising a compound of claim 1 to the subject;
imaging the fluorescence; and
comparing the fluorescence to a control value of a subject without bone erosion.

9. The method of claim 8, wherein the administering of the pharmaceutical composition is selected from the route of administration consisting of intravenous, intradermal, subcutaneous, oral, transdermal, transmucosal, and rectal.

10. The method of claim 8, wherein the pharmaceutical composition further comprises at least one excipient.

11. The method of claim 8, wherein the imaging is visualized by confocal microscopy or near-infrared (NIR) fluorescence imaging systems.

12. A compound selected from the group consisting of Formula II and Formula III, 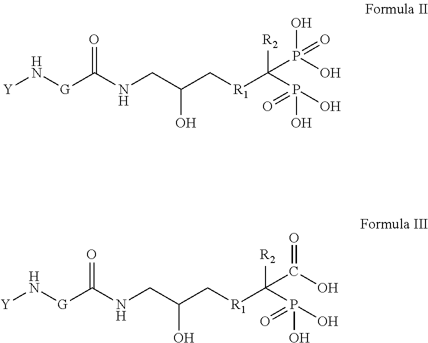
wherein Y is a fluorescent dye,
wherein G is selected from the group consisting of linear, branched, and cyclic alkyl chains, wherein the number of carbon atoms is 1 to 8, and polyethyleneoxy units of formula (OCH₂CH₂)ₘ, wherein m is an integer from 1 to 3000,
wherein R₁ is selected from the group consisting of a 3-ethyl-pyridine substituent or a 1-ethyl-1H-imidazole substituent,
and wherein R₂ is selected from the group consisting of H, OH, F and Cl.

13. The compound of claim 12, wherein Y is selected from the group consisting of 5-carboxyfluorescein (5-FAM), 6-FAM, Alexa Fluor 647 (AF647), and Sulfo-Cyanine5 (Sulfo-Cy5).

14. A method of noninvasively identifying a bone disease or a bone condition in a subject comprising:
administering a pharmaceutical composition comprising a compound of claim 11 to the subject;
measuring the fluorescence in bone tissue or bone cells; and
comparing the fluorescence to a control value of a subject without a bone disease and bone condition.

15. The method of claim 14, wherein the bone disease or bone condition is selected from the group consisting of osteoporosis, Paget's disease, metastatic bone cancers, multiple myeloma, hyperparathyroidism, rheumatoid arthritis, algodystrophy, stemo-costoclavicular hyperostosis, Gaucher's disease and Engleman's disease.

16. The method of claim 14, wherein the administering of the pharmaceutical composition is selected from the route of administration consisting of intravenous, intradermal, subcutaneous, oral, transdermal, transmucosal, and rectal.

17. The method of claim 14, wherein the pharmaceutical composition further comprises at least one excipient.

18. A method of delivering imaging probes to sites of bone erosion comprising:
administering a pharmaceutical composition comprising a compound of claim 11 to the subject;
imaging the fluorescence; and
comparing the fluorescence to a control value of a subject without bone erosion.

19. The method of claim 18, wherein the administering of the pharmaceutical composition is selected from the route of administration consisting of intravenous, intradermal, subcutaneous, oral, transdermal, transmucosal, and rectal.

20. The method of claim 18, wherein the pharmaceutical composition further comprises at least one excipient.

21. The method of claim 18, wherein the imaging is visualized by confocal microscopy or near-infrared (NIR) fluorescence imaging systems.
